(19) United States
(12) Patent Application Publication
(10) Pub. No.: US 2026/0112856 A1
(43) Pub. Date: Apr. 23, 2026

(54) SPINAL CORD STIMULATION DEVICES, SYSTEMS, AND METHODS OF MANUFACTURING

(71) Applicant: NeuroOne Medical Technologies Corporation, Eden Prairie, MN (US)

(72) Inventors: Alfonso Chavez, San Jose, CA (US); Camilo Diaz-Botia, Monte Sereno, CA (US); Maria Francisca Porto Cruz Westermann, Düsseldorf (DE); Mary McNeil, Campbell, CA (US); Maria Vomero, Brooklyn, NY (US); Samuel Ong, San Francisco, CA (US)

(21) Appl. No.: 19/295,133

(22) Filed: Aug. 8, 2025

Related U.S. Application Data
(60) Provisional application No. 63/680,711, filed on Aug. 8, 2024.

(51) Int. Cl. H01R 43/20; A61N 1/05; A61N 1/375

(52) U.S. Cl. CPC H01R 43/20; A61N 1/0551; A61N 1/3752; H01R 2201/12

(57) ABSTRACT

A neuro-stimulation lead having a generally planar stimulation portion and a proximal connector portion for connecting to an implantable pulse generator (“IPG”). A method of forming a neuro-stimulation lead having a generally planar stimulation portion and a proximal connector portion for connecting to an IPG.

### CROSS-REFERENCE TO RELATED APPLICATION(S)

This application claims the benefit under 35 U.S.C. § 119(e) to U.S. Provisional Application 63/680,711, filed Aug. 8, 2024 and entitled “Spinal Cord Stimulation Devices, Systems, and Methods of Manufacturing,” which is hereby incorporated herein by reference in its entirety.

### FIELD

The various embodiments herein relate to stimulation devices and systems for monitoring and/or stimulating the spinal cord and/or peripheral nerves, and related methods of producing such devices and systems.

### BACKGROUND

Electrical stimulation of the spinal cord can result in pain reduction and/or elimination. Medical devices having electrodes (also referred to as “stimulators” or “leads”) are often implanted near the spinal column to provide pain relief for chronic intractable pain. The electrodes stimulate tissue within the spinal column to reduce pain sensations at other parts of the body. The electrodes may stimulate tissue by delivering electrical energy from an energy source (e.g., a battery). The stimulation signals applied can be optimized for pain reduction or elimination depending on the location of the pain.

Thin film spinal cord stimulation devices often comprise a relatively flat or planar electrode body portion at a distal portion of the device for delivering a stimulation signal to tissue within the spinal column of a patient. The energy for producing the stimulation signal is typically provided by a battery housed within an implantable pulse generator. The spinal cord stimulation device is typically placed in electrical communication with the implantable pulse generator (or “IPG”) by inserting the proximal end of the spinal cord stimulation device into an entry port of the IPG. The terminal connection (or proximal connector portion) of the spinal cord stimulation device is typically a cylindrical component configured to be inserted into a generally cylindrical entry port opening located in a header of the IPG, for example. Thus, spinal cord stimulation devices often have a generally flat portion adjacent to a generally cylindrical portion, which can present challenges during the manufacturing process.

There is a need in the art for improved methods of forming electrical connections between spinal cord stimulation devices and implantable pulse generators, and for related systems and devices.

### BRIEF SUMMARY

Discussed herein are various methods of manufacturing certain types of probe devices, and more specifically various methods for forming a proximal connector of a neural or spinal probe device.

In Example 1, a method of manufacturing a proximal connector of a probe device comprises positioning an elongate cylindrical polymeric element adjacent to a flat proximal connector portion of the probe device, along a longitudinal axis of the connector, the flat proximal connector portion comprising a nonconductive substrate having a plurality of conductive proximal contacts disposed thereon, wrapping the flat proximal connector portion around the elongate cylindrical polymeric element to form the proximal connector portion into a generally tubular shape, placing a length of heat shrink tubing over the generally tubular proximal connector portion, applying heat to the heat shrink tubing to shrink the heat shrink tubing onto the generally tubular proximal connector portion, heating the generally tubular proximal connector portion with the heat shrink tubing in an oven, and removing the heat shrink tubing from the plurality of conductive proximal contacts to expose the plurality of conductive proximal contacts.

Example 2 relates to the method according to Example 1, wherein the nonconductive substrate comprises a thin-film material.

Example 3 relates to the method according to Example 2, wherein the thin-film material comprise liquid crystal polymer, polyimide, parylene C, or a combination thereof.

Example 4 relates to the method according to Example 1, further comprising pre-forming the flat proximal connector portion into a semi-cylindrical shape prior to positioning the elongate cylindrical polymeric element adjacent thereto.

Example 5 relates to the method according to Example 4, wherein the pre-forming the flat proximal connector portion into a semi-cylindrical shape comprises inserting the flat proximal connector portion into a lumen of a metal tube such that the proximal connector portion is sufficiently curved to fit within the lumen, and applying heat to the metal tube and the proximal connector portion disposed within the metal tube such that the proximal connector portion is formed into the semi-cylindrical shape.

Example 6 relates to the method according to Example 4, wherein the pre-forming the flat proximal connector portion into a semi-cylindrical shape comprises positioning a cylindrical core adjacent to the flat proximal connector portion, and wrapping the proximal connector portion around the cylindrical core such that the proximal connector portion is formed into the semi-cylindrical shape.

Example 7 relates to the method according to Example 1, wherein the heating in the oven is performed at a temperature of about 200 degrees Celsius.

Example 8 relates to the method according to Example 1, wherein the heating in the oven is performed for a time period ranging from about 10 minutes to about 20 minutes.

Example 9 relates to the method according to Example 1, further comprising disposing a marker band on the proximal connector portion after forming the generally tubular shape.

Example 10 relates to the method according to Example 1, wherein the plurality of conductive proximal contacts comprise platinum, platinum iridium, iridium oxide, titanium, or combinations thereof.

In Example 11, a method of manufacturing a neural or spinal probe device comprises pre-forming a flat proximal connector portion of the probe device into a semi-cylindrical shape, the flat proximal connector portion comprising a nonconductive substrate having a plurality of conductive proximal contacts disposed thereon, positioning an elongate cylindrical polymeric element within the partial lumen of the semi-cylindrical proximal connector portion, wrapping the semi-cylindrical proximal connector portion around the elongate cylindrical polymeric element to form the proximal connector portion into a generally tubular shape, placing a length of heat shrink tubing over the generally tubular proximal connector portion, applying heat to the heat shrink tubing to shrink the heat shrink tubing onto the generally tubular proximal connector portion, heating the generally tubular proximal connector portion with the heat shrink tubing in an oven, and removing the heat shrink tubing from the plurality of conductive proximal contacts to expose the plurality of conductive proximal contacts.

Example 12 relates to the method according to Example 11, further comprising coupling the proximal connector portion to a controller/power source device.

Example 13 relates to the method according to Example 11, wherein the step of pre-forming the flat proximal connector portion into a semi-cylindrical shape comprises placing the flat proximal connector portion adjacent to a semi-rigid cylindrical core and wrapping the connector portion around the core to form the semi-cylindrical shape.

Example 14 relates to the method according to Example 11, wherein the step of pre-forming the flat proximal connector portion into a semi-cylindrical shape comprises inserting the flat proximal connector portion into a lumen of an elongate metal tube and applying heat to the metal tube to set the connector portion in the semi-cylindrical shape.

Example 15 relates to the method according to Example 11, wherein the nonconductive substrate comprises a thin-film material.

Example 16 relates to the method according to Example 15, wherein the thin-film material comprises a material selected from the group consisting of liquid crystal polymer, polyimide, and parylene C.

Example 17 relates to the method according to Example 11, further comprising positioning a marker band on the proximal connector portion after forming the generally tubular shape.

In Example 18, a method of manufacturing a neural or spinal probe device comprises obtaining a flat proximal connector portion of the neural or spinal probe device, the flat proximal connector portion comprising a nonconductive substrate and a plurality of conductive proximal contacts disposed thereon, pre-forming the flat proximal connector portion into a semi-cylindrical shape by either (i) wrapping the connector portion around a semi-rigid cylindrical core or (ii) inserting the connector portion into a lumen of an elongate metal tube and applying heat, positioning an elongate cylindrical polymeric element within the partial lumen of the semi-cylindrical proximal connector portion, wrapping the semi-cylindrical proximal connector portion around the elongate cylindrical polymeric element to form the proximal connector portion into a generally tubular shape, placing a length of heat shrink tubing over the generally tubular proximal connector portion, applying heat to the heat shrink tubing to shrink the heat shrink tubing onto the generally tubular proximal connector portion, heating the generally tubular proximal connector portion with the heat shrink tubing in an oven, removing the heat shrink tubing from the plurality of conductive proximal contacts to expose the plurality of conductive proximal contacts, and coupling the proximal connector portion to a controller or power source device.

Example 19 relates to the method according to Example 18, wherein the nonconductive substrate comprises a material selected from the group consisting of liquid crystal polymer (LCP), polyimide, and parylene C.

Example 20 relates to the method according to Example 18, further comprising disposing a marker band on the proximal connector portion after forming the generally tubular shape.

While multiple embodiments are disclosed, still other embodiments will become apparent to those skilled in the art from the following detailed description, which shows and describes various illustrative implementations. As will be realized, the various embodiments herein are capable of modifications in various obvious aspects, all without departing from the spirit and scope thereof. Accordingly, the drawings and detailed description are to be regarded as illustrative in nature and not restrictive.

### BRIEF DESCRIPTION OF THE DRAWINGS

FIG. 1A is a partial top view of a neuro-stimulation device formed according to an embodiment of this disclosure;

FIG. 1B is a partial, side view of a probe device formed according to an embodiment of this disclosure;

FIG. 1C is a partial, perspective view of a probe device formed according to an embodiment of this disclosure;

FIG. 2 is a schematic view of a probe device with a connector portion inserted into a header of an IPG, according to an embodiment of this disclosure;

FIG. 3A is a schematic plan view of a probe device according to an embodiment of this disclosure;

FIG. 3B is a schematic plan view of a probe device according to an embodiment of this disclosure;

FIG. 4A is enlarged top view of a connector portion of a probe device during manufacturing according to an embodiment of this disclosure;

FIG. 4B is an enlarged side view of an end of the device of FIG. 4A, according to one embodiment;

FIG. 4C is enlarged schematic view of the connector portion of the probe device of FIG. 4A with a core disposed adjacent to the device during manufacturing according to an embodiment of this disclosure;

FIG. 4D is an enlarged side view of an end of the device of FIG. 4C, according to one embodiment;

FIG. 4E is enlarged schematic view of a connector portion of a probe device during manufacturing according to an embodiment of this disclosure;

FIG. 4F is enlarged schematic view of a connector portion of a probe device during manufacturing according to an embodiment of this disclosure;

FIG. 5A is an enlarged schematic view of the connector portion of a probe device being inserted into a header of an IPG according to an embodiment of this disclosure;

FIG. 5B is an enlarged schematic view of the connector portion of a probe device being inserted into a header of an IPG according to an embodiment of this disclosure;

FIG. 6A is a flow diagram showing a series of method steps for forming a probe device according to an embodiment of this disclosure;

FIG. 6B is a flow diagram showing a series of method steps for forming a probe device according to another embodiment;

FIG. 7A is a schematic view of a probe device being manufactured according to an embodiment of this disclosure;

FIG. 7B is a schematic cross-sectional end view of a probe device being manufactured according to an embodiment of this disclosure;

FIG. 7C is a schematic cross-sectional end view of a probe device being manufactured according to an embodiment of this disclosure;

FIG. 8A is a schematic perspective view of a probe device being formed with a marker band according to an embodiment of this disclosure;

FIG. 8B is a schematic top view of a probe device being formed with a marker band according to an embodiment of this disclosure;

FIG. 8C is a schematic top plan view of a Pellethane core used in forming a probe device according to an embodiment of this disclosure;

FIG. 9A is a schematic side view of a connector portion of a probe device according to an embodiment of this disclosure;

FIG. 9B is a schematic side view of a connector portion of a probe device according to an embodiment of this disclosure; and

### DETAILED DESCRIPTION

The various embodiments disclosed or contemplated herein relate to neural or spinal probes, including detection, stimulation, and ablation probes and devices, and improved systems, devices, and methods thereof. In certain exemplary implementations, there are manufacturing methods provided for forming such devices that incorporate thin-film technology. More specifically, methods are provided for making either or both neural and/or spinal probe devices such as the exemplary devices depicted and described herein. The various devices disclosed or contemplated herein relate to a neural or spinal probe device having an improved proximal (or “terminal”) connector for coupling to an external device or an internal device such as an implantable pulse generator (“IPG”) or similar devices for powering and controlling such neural or spinal probe devices. More specifically, such proximal connectors can couple to a header portion or other connection portion of such external or internal devices such as IPGs or the like.

The exemplary types of thin film probes that could incorporate the various embodiments disclosed or contemplated herein can include, but are not limited to, a cortical electrode device, a depth electrode device, a spinal cord stimulation device, or any other known neural or spinal probes. Further, any such neural or spinal probes˜in addition to having a proximal connector as disclosed herein˜can also have any other known features or structures of known neural or spinal probes.

For purposes of this application, any of the various device embodiments herein can be referred to interchangeably as a “probe,” “probe device,” “electrode,” “electrode device, “lead,” or “device lead.” Any of these terms can be used to describe any neural or spinal electrode device that can be used for recording, ablation, and/or stimulation.

In many applications, a probe device will comprise multiple conductive elements that extend along a length of the device. As is understood with such neural and spinal probes, each such conductive element terminates at the distal end with electrode contacts for use at the target site and at the proximal end with exposed terminal or proximal contacts configured to make electrical contact with separate electrical connections within the power/control device, such as the header of an IPG (or other comparable connector type) as noted above. For example, in neuro-stimulation applications, a probe device may have as many as 12 or more conductors and thus 12 proximal contacts, with corresponding connections in the power/control device.

Disclosed herein are improvements to proximal connectors for probe devices, for example, probe devices used in neurostimulation applications or the like. In some embodiments, an improved proximal connector for a probe device, and/or a method of manufacturing such a proximal connector, are disclosed. A terminal connector portion of the device is configured to be inserted into a port typically found in an external device such as an IPG (or a header of the IPG) to operably couple the IPG to the device. This enables the IPG or other controller/power source device to (a) receive signals from contacts at a distal end of the probe device (e.g., to monitor, sense, and detect electrical signals indicative of physiological parameters of a patient, and (b) send electrical signals to an area of a patient's anatomy to treat and/or address a sensed condition, for example. In other embodiments, it enables the external controller/power source to send electrical energy to a target area for ablation. In some cases, the IPG or other controller/power source may include software and/or microprocessors configured to determine when and how to deliver a therapeutic stimulation signal in response to the signals received from contacts at the distal end of the medical device lead.

In certain exemplary embodiments, the various proximal connector embodiments disclosed or contemplated herein can couple with a header portion of an IPG that is a commercially available component called a Bal Conn® electrical connector, which is available from Bal Seal Engineering. Alternatively, the IPG or other external controller/power source device can have any known port or connection assembly to couple with any of the proximal connector implementations herein.

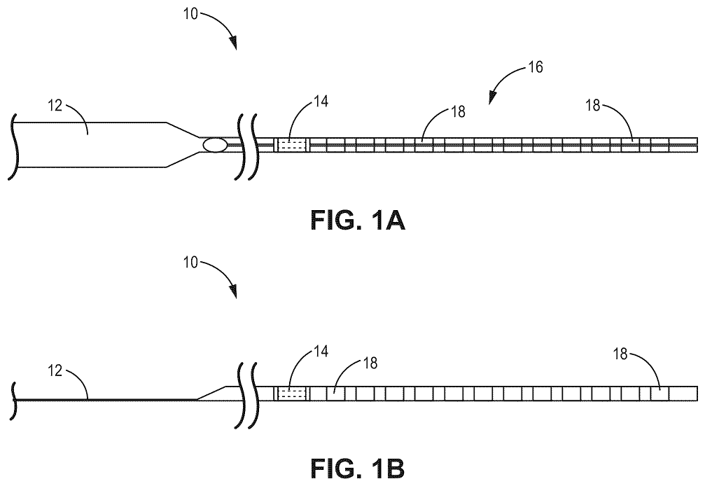

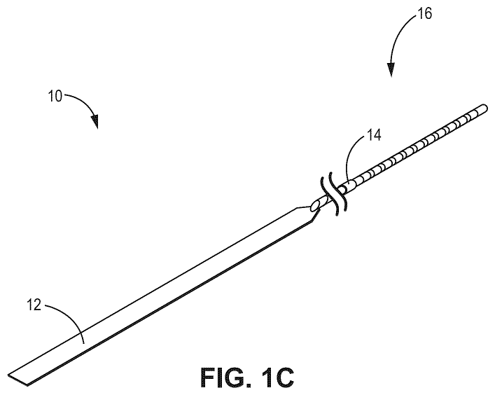

FIG. 1A is a partial, top view, FIG. 1B is a partial, side view, and FIG. 1C is a partial, perspective view of a probe device 10 according to embodiments of this disclosure. The probe device 10 may, for example, be a percutaneous spinal cord stimulation device (also referred to as a “linear device”) for stimulating the spinal cord or related peripheral nerves in the human body. Alternatively, the probe device 10 can be any known neural or spinal probe device. In certain implementations, the device 10 may incorporate thin-film technology. In this particular embodiment as shown, and in the various other figures herein, it is understood that the length of the various device embodiments between the electrode contact pad 12 and the proximal connector section 16 can be far longer than is shown schematically in the figures.

As shown in FIGS. 1A-1C, the probe device 10 may include an electrode contact pad 12 and a proximal connector section 16 with a length of the probe device body therebetween (not shown), generally as shown in FIGS. 1A-1C. The proximal connector 16 has multiple proximal contacts 18 disposed along the length of the proximal connector 16 as shown. The electrode contact pad 12 is disposed distal to the proximal connector portion 16; in some cases, electrode contact pad 12 may form a distal portion of the probe device 10. The contact pad 12 may include two or more electrode contacts or electrical contacts (not shown) disposed on the pad 12 for stimulating, sensing, ablation, and the like at the target site in the patient. In some cases, the electrode contacts constitute a conductive surface exposed at a surface of the pad 12, and each electrode contact may be electrically coupled to a corresponding proximal contact within the proximal connector portion 16 via an elongate conductor (not shown) that extends therebetween. In some implementations, as many as 12 or more contacts may be disposed on the pad 12. The pad 12 may be generally planar or flat when in use to facilitate placement of the associated contacts in contact with a generally planar surface of a patient's anatomy. In some cases, the pad 12 may be configured to fold or curl around a generally longitudinal axis of the pad 12 to facilitate deployment to a particular location in a human body; upon placement, the pad 12 may be configured to un-fold or un-furl into a generally planar configuration.

In some embodiments, the probe device 10 may also include a marker band 14 disposed on the proximal connector portion 16 of device 10; as shown in FIGS. 1A-1C, marker band 14 may be disposed at a distal end of the proximal connector portion 16, although the precise location may be varied. Marker band 14 may be used to facilitate locating and/or guiding the placement of the probe device 10 during implantation of device 10 in a patient, for example. That is, marker band 14 may be formed of a radiopaque material and/or provide visual indicators that are visible via various medical visualization technologies, including X-ray fluoroscopy or any other known imaging technology. In certain embodiments, the marker band 14 may additionally serve as a somewhat rigid structure or support for holding the curled up proximal connector portion 16 in a desired shape (e.g., having a generally circular cross-sectional profile), e.g., during the manufacturing process. Further, marker band 14 may also be used to help form a physical connection of the probe device 10 to an external controller/power source such as an IPG, or to maintain the positioning of the device 10 with respect to the external controller/power source (not shown in FIGS. 1A-1C).

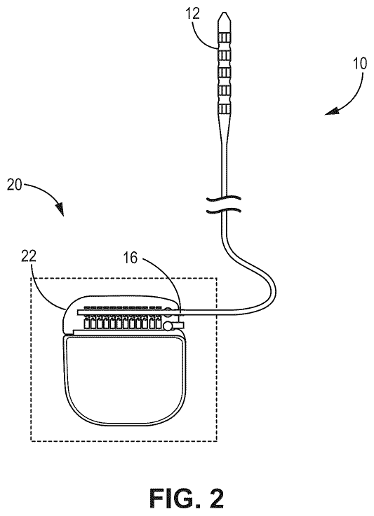

FIG. 2 is a schematic view of the probe device 10 with the proximal connector portion 16 inserted into a header 22 or header portion 22 of an IPG 20. The individual electrode contacts 18 along proximal connector portion 16 make electrical connections within header portion 22 to establish conduction paths to electrode body portion 12 of device 10. Alternatively, the proximal connector portion 16 can be inserted into and thereby coupled to any similar controller/power source device.

The various non-conducting thin-film components of the device 10 (and any other device embodiment herein)—such as the base layer, etc.—can be made of polyimide (“PI”), parylene C, liquid crystal polymer (“LCP”), or similar materials. The conductive materials used in the device 10 (for the contacts, for example) can be any one or more of platinum, platinum iridium, iridium oxide, titanium, or any other known conductive metal for use in spinal or neural probe devices.

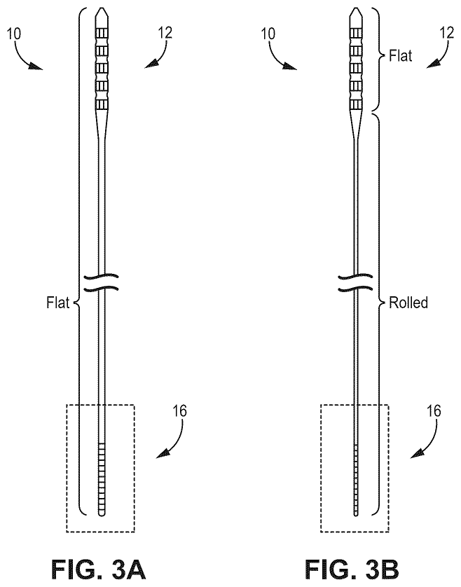

FIGS. 3A and 3B are schematic plan views of a probe device 10 during the manufacturing process. FIG. 3A shows the device 10 with the proximal connector portion 16 in a “flat” configuration during manufacturing, according to some embodiments. FIG. 3B shows neuro-stimulation device 10 with the proximal connector portion 16 in a curved, or generally cylindrical, or “rolled” configuration upon completion of manufacturing, according to some embodiments.

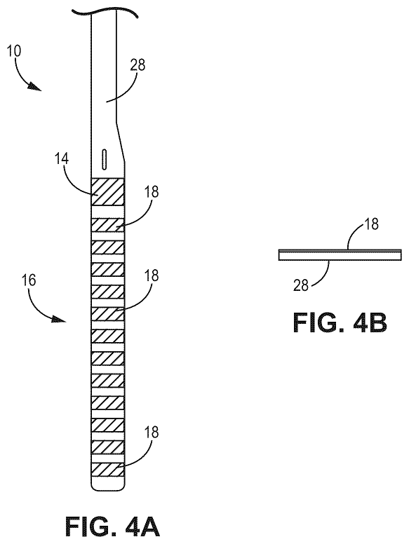

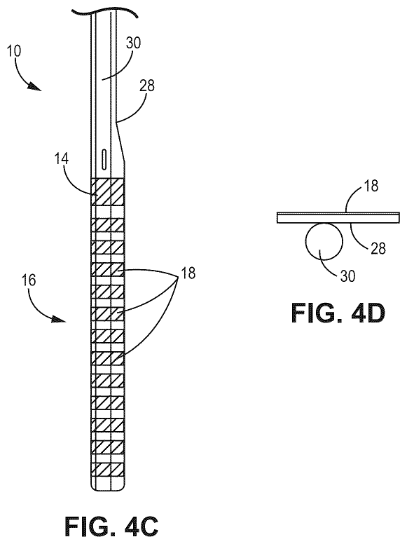

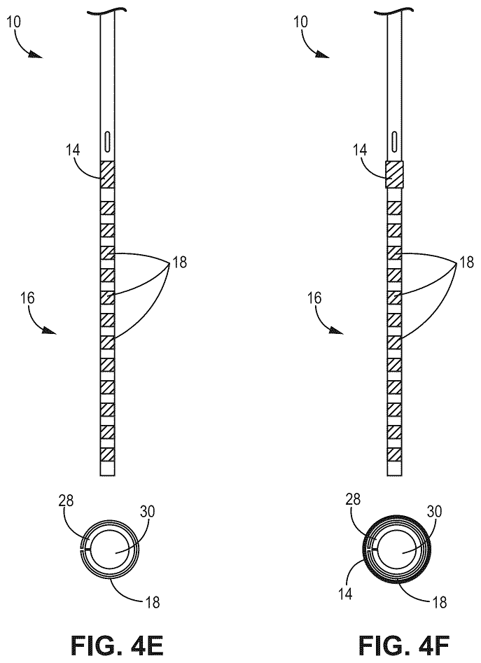

One embodiment of the process of making a proximal connector will now be discussed in additional detail. FIGS. 4A and 4B are enlarged schematic views of the proximal connector portion 16 of the device 10 in a “flat” configuration during manufacturing (before the formation of the rod-like or tube-like shape). FIG. 4A illustrates the exemplary device 10 having twelve proximal contacts 18 disposed along the length of proximal connector portion 16. An exemplary location of marker band 14 is also depicted in FIG. 4A, although the exact location can be varied along proximal connector portion 16 according to various embodiments. As shown in both FIG. 4A and FIG. 4B, the device 10 has a non-conductive base layer or “substrate” 28 with the conductive proximal contact material 18 disposed thereon. According to one embodiment, the base layer 28 is made of a liquid crystal polymer (“LCP”). Alternatively, the base layer 28 can be made of any thin-film material or any other material.

Prior to forming the proximal connector 16 into its tube-like shape, an elongate polymeric core 30 is disposed next to and in contact with the connector 16, as best shown in FIGS. 4C and 4D. More specifically, FIGS. 4C and 4D illustrate the placement of the elongate core 30 along a longitudinal axis of the proximal connector portion 16. In some embodiments, the core 30 is made of a polymeric material such as Pellethane 55. Alternatively, any known polymeric material for use as a core of a probe device can be used. In some embodiments, the substrate 28 of the proximal connector portion 16 can be pre-curved or pre-rolled to give it a curved or somewhat cylindrical shape prior to placement or insertion of the core 30 thereon (or therein). In such cases, as will be discussed in additional detail below, a semi-rigid cylindrical core material (e.g., a polyimide core) may be used to pre-form or shape the proximal connector portion 16 into a partially rolled configuration prior to insertion of the core 30, for example.

Once the core 30 is placed as desired (as discussed above), the flat proximal connector 16 is then wrapped around the core 30 to form the tube-like shape of the connector 16 as shown in FIGS. 4E and 4F, which show the “rolled” or cylindrical configuration of the proximal connector portion 16 during manufacturing of device 10. More specifically, FIG. 4E shows the substrate 28 of device 10—and the proximal contacts 18—having been wrapped around to conform to the generally cylindrical shape of the core 30. FIG. 4F shows the further step of placing marker band 14 on proximal connector portion 16. In some embodiments, marker band 14 may be generally cylindrical and may be used to hold the substrate 28 of device 10 in a cylindrical, rolled shape, according to some implementations.

As shown in the exemplary configuration shown in FIG. 4F, marker band 14 may disposed on proximal connector portion 16 of device 10 at a distal portion thereof; that is, marker band 14 may be disposed distally of the electrode contacts 18 according to the depicted embodiment, although this is not the only configuration contemplated. In some embodiments, marker band 14 may have a slightly larger outer radius than the proximal contacts 18; this could, for example, facilitate placement and/or “seating” of the proximal connector portion 16 within header portion 22 of IPG 20 (or a port or other lumen of any such controller/power source device) during implantation in a patient, for example. In further embodiments, marker band 14 may also serve as a set screw engagement feature, for example, to enable a set screw in the header 22 of an IPG 20 or equivalent device to be tightened onto marker band 14 to hold proximal connector portion 16 in place in header 22. This may further help to ensure proper positioning such that electrode contacts 18 are positioned in header 22 to make contact with respective connectors of the IPG 20 or other such device.

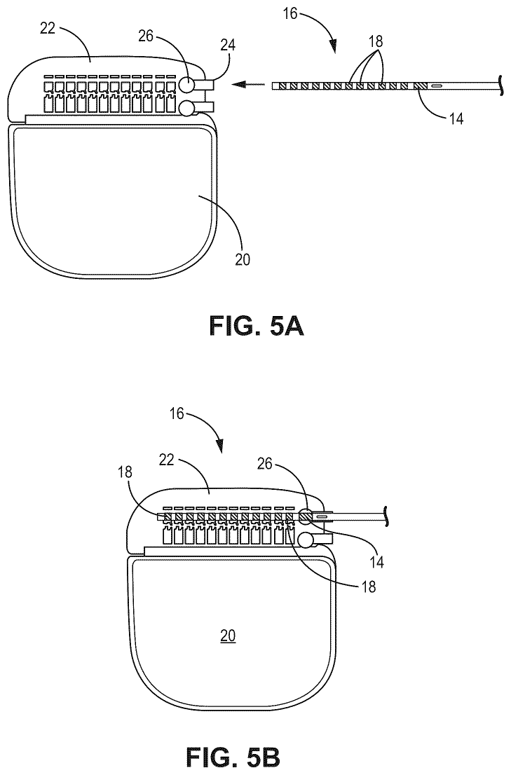

According to one exemplary version, FIGS. 5A and 5B are enlarged schematic views of the proximal connector portion 16 of device 10 being inserted into a header 22 of an IPG 20. For example, FIG. 5A shows proximal connector portion 16 of device 10 positioned proximate to an entry port 24 of IPG 20. FIG. 5B shows proximal connector portion 16 fully inserted into header 22 of IPG 20 via entry port 24. FIG. 5B also shows the use of a set screw 26 (typically part of header 22 of IPG 20) to securely engage with marker band 14 of device 10 to hold proximal connector portion 16 in place and/or to ensure establishment and maintenance of electrical conduction paths at each of the proximal contacts 18.

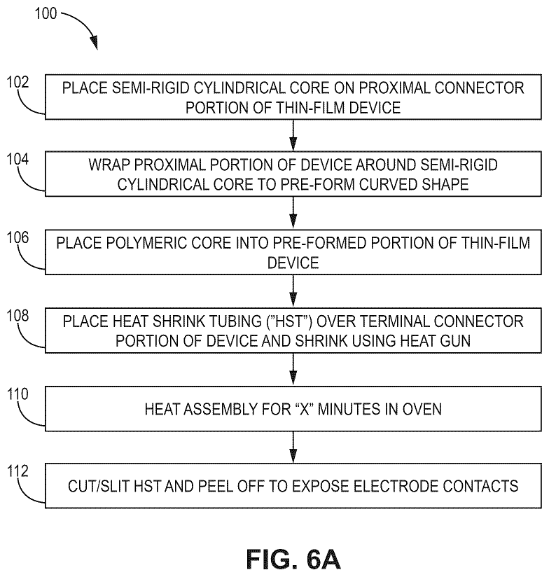

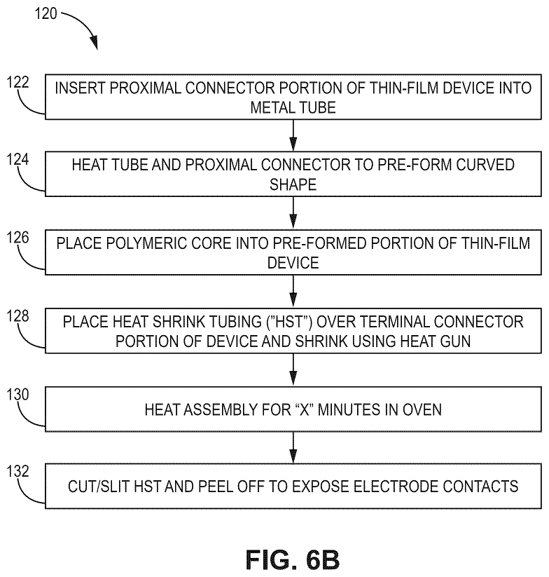

One exemplary method for manufacturing a proximal connector 100 as contemplated herein is set forth in FIG. 6A. More specifically, FIG. 6A is a flow diagram showing a series of exemplary method steps for forming the probe device 10 with proximal connector portion 16, according to various embodiments of this disclosure. First, the proximal connector 16 can optionally be formed into a semi-circular or curved shape as mentioned above. That is, one optional initial step is to place a semi-rigid cylindrical core adjacent to and in contact with the proximal connector portion (such as connector 16) (102). Next, the proximal connector section (such as connector 16) can be wrapped around the cylindrical core to pre-form the connector into a generally tubular or cylindrical shape (104).

The next step—or the first step if the connector is not pre-formed into an at least semi-cylindrical shape—is to place a core (such as core 30) along the device 10 in a fashion such as that depicted in FIGS. 4C-4D (106). (In those embodiments where the proximal portion has been pre-formed, this step may involve placing the core into the “rolled” portion of the proximal connector section.)

Once the core 30 is disposed next to the connector 16, the flat connector 16 is then wrapped around the core 30 as best shown in FIGS. 4E and 4F.

Once the connector 16 is formed into the tubular or cylindrical shape, the next step can be to place a length of heat shrink tubing (“HST”) over the proximal connector portion of the device and apply heat to the HST via a heat gun or other device or method to shrink the HST around the proximal connector 16 (108).

After shrinking the HST onto the connector (such as connector 16), the device assembly can be heated in an oven (110). In some embodiments, the time for heating the device assembly in the oven may be 15 minutes, although the exact time for heating may vary depending on a number of factors and could be more or less than 15 minutes. For example, the time could range from 8 minutes to 22 minutes. Alternatively, it could range from 10 to 20 minutes. In some embodiments, the temperature at which the device assembly is heated in the oven may be approximately 200 degrees Celsius, although the precise temperature for heating the device assembly may vary depending on a number of factors and could be more or less than 200 C.

Once the assembly has been heated in the oven, the device can be removed and the heat shrink tubing or material can be removed (such as by cutting or slitting) from the proximal contacts to expose those contacts for use (112).

Another exemplary method for manufacturing a proximal connector 120 as contemplated herein is set forth in FIG. 6B. More specifically, FIG. 6B is a flow diagram showing a series of exemplary method steps for forming the probe device 10 with proximal connector portion 16, according to various embodiments of this disclosure. First, the flat proximal connector 16 can inserted into the lumen of an elongate metal tube (122). That is, the proximal end of the connector 16 can be squeezed or otherwise deformed into a curved shape to fit it into the end of the metal tube such that the connector 16 can then be urged therein. Once the connector is inserted into the tube as desired, heat is applied to the tube (and the connector disposed therein), thereby causing the proximal connector to be set in a preformed curved shape (124). The connector can then be removed from the tube.

The next step—or the first step if the connector is not pre-formed into an at least semi-cylindrical shape as described above—is to place a core (such as core 30) along the device 10 in a fashion such as that depicted in FIGS. 4C-4D (126). (In those embodiments where the proximal portion has been pre-formed, this step may involve placing the core into the “rolled” portion of the proximal connector section.) Once the core 30 is disposed next to the connector 16, the flat connector 16 is then wrapped around the core 30 as best shown in FIGS. 4E and 4F.

Once the connector 16 is formed into the tubular or cylindrical shape, the next step can be to place a length of heat shrink tubing (“HST”) over the proximal connector portion of the device and apply heat to the HST via a heat gun or other device or method to shrink the HST around the proximal connector 16 (128).

After shrinking the HST onto the connector (such as connector 16), the device assembly can be heated in an oven (130). In some embodiments, the time for heating the device assembly in the oven may be 15 minutes, although the exact time for heating may vary depending on a number of factors and could be more or less than 15 minutes. For example, the time could range from 8 minutes to 22 minutes. Alternatively, it could range from 10 to 20 minutes. In some embodiments, the temperature at which the device assembly is heated in the oven may be approximately 200 degrees Celsius, although the precise temperature for heating the device assembly may vary depending on a number of factors and could be more or less than 200 C.

Once the assembly has been heated in the oven, the device can be removed and the heat shrink tubing or material can be removed (such as by cutting or slitting) from the proximal contacts to expose those contacts for use (132).

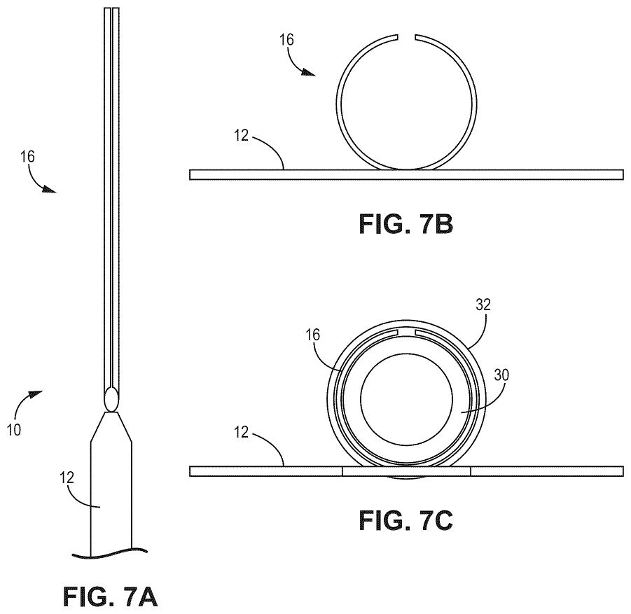

FIGS. 7A-9B are a series of schematic views illustrating some of the steps of the above-described method, according to one embodiment. FIG. 7A, for example, shows a portion of a probe device 10 in which the proximal connector portion 16 of device 10 has been pre-formed into a generally cylindrical shape. FIGS. 7B and 7C show cross-sectional end views of device 10. For example, FIG. 7B shows the generally cylindrical shape of proximal connector portion 16 after being pre-formed according to steps 102, 104 or steps 122, 124 as discussed above, depending on the method used. FIG. 7C shows the placement of the core 30 within a lumen formed by shaping the proximal connector portion 16 into a “rolled” or generally cylindrical shape. FIG. 7C also illustrates the placement and positioning of heat shrink tubing or material 32 over the proximal connector portion 16.

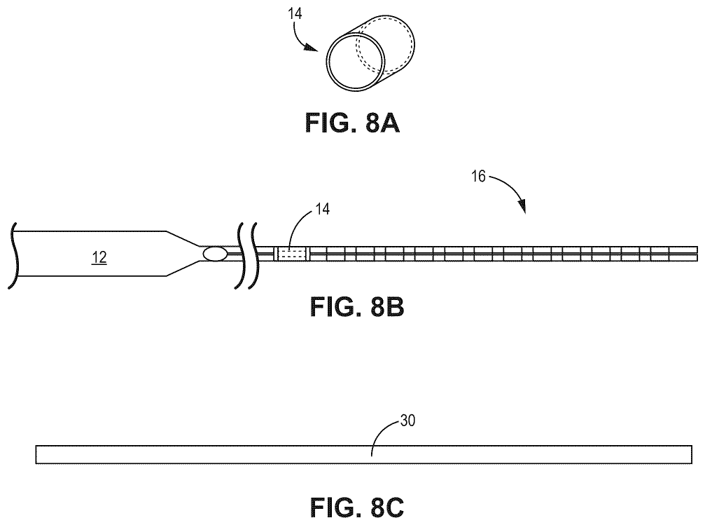

FIG. 8A is a perspective view of an exemplary marker band 14 that may be used in forming device 10. FIG. 8B is a top view of device 10 showing an exemplary placement and/or positioning for marker band 14 according to some embodiments. Marker band 14 may be formed of a radiopaque material such that it may be readily located during x-ray fluoroscopy to provide an operator with visual information that may be helpful in navigating device 10 to a desired anatomical location in a patient, for example. In some embodiments, marker band 14 may additionally, or alternatively, be formed of a material of suitable strength for forming a mechanical engagement of the proximal connector portion 16 within the header 22 of IPG 20 via a set screw 26, as discussed above. For example, when proximal connector portion 16 is fully inserted in header 22, the placement of marker band 14 may be such that it aligns with the location of a set screw 26; tightening of the set screw 26 when the proximal connector portion 16 is positioned within the header 22 of IPG 20 (or equivalent device) may thereby form a secure mechanical connection between the device 10 and the IPG 20, and may also function to position each of the plurality of electrode contacts 18 of device 10 in electrical contact with each of the corresponding connectors within the header 22 of IPG 20. FIG. 8C is a plan view of the core 30.

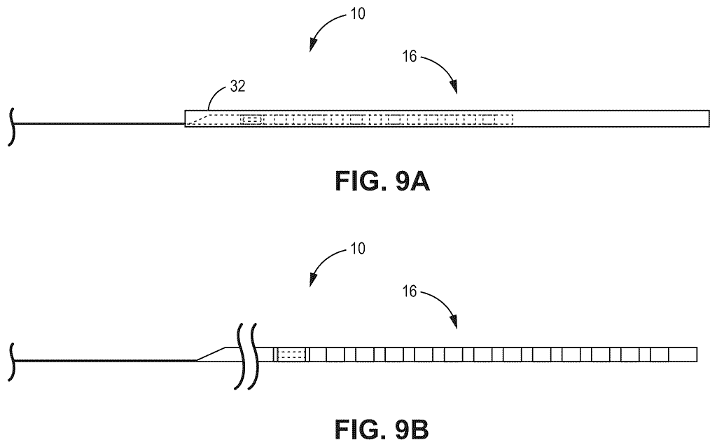

FIGS. 9A and 9B are schematic side views of proximal connector portion 16 of device 10. FIG. 9A illustrates the placement of heat shrink material or tubing 32 over the proximal connector portion 16 of device 10. FIG. 9B illustrates the resultant proximal connector portion 16 following cutting and/or slitting of the HST (after heating to shrink the HST) and removing (e.g., peeling off) HST to expose the electrode contacts 18 disposed along the proximal connector portion 16 of device 10.

It should be noted that the manufacturing methods disclosed and described herein may be equally applicable to any types of neural or spinal probe devices, including, for example, spinal cord stimulation devices having “paddle arrays,” for example, that have a width that may require the paddle leads to be curled or wound or otherwise contracted during implantation, and which are subsequently unfurled and/or fully deployed upon positioning in a desired anatomical location of interest.

While the various systems described above are separate implementations, any of the individual components, mechanisms, or devices, and related features and functionality, within the various system embodiments described in detail above can be incorporated into any of the other system embodiments herein.

The terms “about” and “substantially,” as used herein, refers to variation that can occur (including in numerical quantity or structure), for example, through typical measuring techniques and equipment, with respect to any quantifiable variable, including, but not limited to, mass, volume, time, distance, wave length, frequency, voltage, current, and electromagnetic field. Further, there is certain inadvertent error and variation in the real world that is likely through differences in the manufacture, source, or precision of the components used to make the various components or carry out the methods and the like. The terms “about” and “substantially” also encompass these variations. The term “about” and “substantially” can include any variation of 5% or 10%, or any amount—including any integer—between 0% and 10%. Further, whether or not modified by the term “about” or “substantially,” the claims include equivalents to the quantities or amounts.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾ This applies regardless of the breadth of the range. Although the various embodiments have been described with reference to preferred implementations, persons skilled in the art will recognize that changes may be made in form and detail without departing from the spirit and scope thereof.

Although the various embodiments have been described with reference to preferred implementations, persons skilled in the art will recognize that changes may be made in form and detail without departing from the spirit and scope thereof.

What is claimed is:

1. A method of manufacturing a proximal connector of a probe device, the method comprising:
positioning an elongate cylindrical polymeric element adjacent to a flat proximal connector portion of the probe device, along a longitudinal axis of the connector, the flat proximal connector portion comprising a nonconductive substrate having a plurality of conductive proximal contacts disposed thereon;
wrapping the flat proximal connector portion around the elongate cylindrical polymeric element to form the proximal connector portion into a generally tubular shape;
placing a length of heat shrink tubing over the generally tubular proximal connector portion;
applying heat to the heat shrink tubing to shrink the heat shrink tubing onto the generally tubular proximal connector portion;
heating the generally tubular proximal connector portion with the heat shrink tubing in an oven; and
removing the heat shrink tubing from the plurality of conductive proximal contacts to expose the plurality of conductive proximal contacts.

2. The method of claim 1, wherein the nonconductive substrate comprises a thin-film material.

3. The method of claim 2, wherein the thin-film material comprise liquid crystal polymer, polyimide, parylene C, or a combination thereof.

4. The method of claim 1, further comprising pre-forming the flat proximal connector portion into a semi-cylindrical shape prior to positioning the elongate cylindrical polymeric element adjacent thereto.

5. The method of claim 4, wherein the pre-forming the flat proximal connector portion into a semi-cylindrical shape comprises:
inserting the flat proximal connector portion into a lumen of a metal tube such that the proximal connector portion is sufficiently curved to fit within the lumen; and
applying heat to the metal tube and the proximal connector portion disposed within the metal tube such that the proximal connector portion is formed into the semi-cylindrical shape.

6. The method of claim 4, wherein the pre-forming the flat proximal connector portion into a semi-cylindrical shape comprises:
positioning a cylindrical core adjacent to the flat proximal connector portion; and
wrapping the proximal connector portion around the cylindrical core such that the proximal connector portion is formed into the semi-cylindrical shape.

7. The method of claim 1, wherein the heating in the oven is performed at a temperature of about 200 degrees Celsius.

8. The method of claim 1, wherein the heating in the oven is performed for a time period ranging from about 10 minutes to about 20 minutes.

9. The method of claim 1, further comprising disposing a marker band on the proximal connector portion after forming the generally tubular shape.

10. The method of claim 1, wherein the plurality of conductive proximal contacts comprise platinum, platinum iridium, iridium oxide, titanium, or combinations thereof.

11. A method of manufacturing a neural or spinal probe device, the method comprising:
pre-forming a flat proximal connector portion of the probe device into a semi-cylindrical shape, the flat proximal connector portion comprising a nonconductive substrate having a plurality of conductive proximal contacts disposed thereon;
positioning an elongate cylindrical polymeric element within the partial lumen of the semi-cylindrical proximal connector portion;
wrapping the semi-cylindrical proximal connector portion around the elongate cylindrical polymeric element to form the proximal connector portion into a generally tubular shape;
placing a length of heat shrink tubing over the generally tubular proximal connector portion;
applying heat to the heat shrink tubing to shrink the heat shrink tubing onto the generally tubular proximal connector portion;
heating the generally tubular proximal connector portion with the heat shrink tubing in an oven; and
removing the heat shrink tubing from the plurality of conductive proximal contacts to expose the plurality of conductive proximal contacts.

12. The method of claim 11, further comprising coupling the proximal connector portion to a controller/power source device.

13. The method of claim 11, wherein the step of pre-forming the flat proximal connector portion into a semi-cylindrical shape comprises placing the flat proximal connector portion adjacent to a semi-rigid cylindrical core and wrapping the connector portion around the core to form the semi-cylindrical shape.

14. The method of claim 11, wherein the step of pre-forming the flat proximal connector portion into a semi-cylindrical shape comprises inserting the flat proximal connector portion into a lumen of an elongate metal tube and applying heat to the metal tube to set the connector portion in the semi-cylindrical shape.

15. The method of claim 11, wherein the nonconductive substrate comprises a thin-film material.

16. The method of claim 15, wherein the thin-film material comprises a material selected from the group consisting of liquid crystal polymer, polyimide, and parylene C.

17. The method of claim 11, further comprising positioning a marker band on the proximal connector portion after forming the generally tubular shape.

18. A method of manufacturing a neural or spinal probe device, the method comprising:
obtaining a flat proximal connector portion of the neural or spinal probe device, the flat proximal connector portion comprising a nonconductive substrate and a plurality of conductive proximal contacts disposed thereon;
pre-forming the flat proximal connector portion into a semi-cylindrical shape by either
(i) wrapping the connector portion around a semi-rigid cylindrical core or
(ii) inserting the connector portion into a lumen of an elongate metal tube and applying heat;
positioning an elongate cylindrical polymeric element within the partial lumen of the semi-cylindrical proximal connector portion;
wrapping the semi-cylindrical proximal connector portion around the elongate cylindrical polymeric element to form the proximal connector portion into a generally tubular shape;
placing a length of heat shrink tubing over the generally tubular proximal connector portion;
applying heat to the heat shrink tubing to shrink the heat shrink tubing onto the generally tubular proximal connector portion;
heating the generally tubular proximal connector portion with the heat shrink tubing in an oven;
removing the heat shrink tubing from the plurality of conductive proximal contacts to expose the plurality of conductive proximal contacts; and
coupling the proximal connector portion to a controller or power source device.

19. The method of claim 18, wherein the nonconductive substrate comprises a material selected from the group consisting of liquid crystal polymer (LCP), polyimide, and parylene C.

20. The method of claim 18, further comprising disposing a marker band on the proximal connector portion after forming the generally tubular shape.
